(19) United States
(12) Patent Application Publication
(10) Pub. No.: US 2023/0077325 A1
(43) Pub. Date: Mar. 16, 2023

(54) NEBULIZER ASSEMBLY AND AIRFLOW-GUIDING COMPONENT THEREOF

(71) Applicant: HCMed Innovations Co., LTD., Taipei City (TW)

(72) Inventors: Wen-Yu Tsai, Taipei City (TW); CHIA-CHIEN CHANG, New Taipei City (TW)

(21) Appl. No.: 17/565,386

(22) Filed: Dec. 29, 2021

(30) Foreign Application Priority Data
Sep. 13, 2021 (TW) 110133990

(51) Int. Cl. A61M 16/14

(52) U.S. Cl. CPC A61M 16/14; A61M 2206/20

(57) ABSTRACT

A nebulizer assembly and an airflow-guiding component thereof are provided. The airflow-guiding component includes an air-guiding part and a structural matching part. The air-guiding part includes a first hollow tube body, a second hollow tube body, and an air-guiding structure connected between the first hollow tube body and the second hollow tube body. The structural matching part is connected to the first hollow tube body. The air-guiding structure has a plurality of rear main air-introducing channels surrounding the first hollow tube body, and each of the plurality of rear main air-introducing channels has a rear main air-introducing opening perpendicular or inclined to the structural matching part. Therefore, external air outside the airflow-guiding component can be guided into the rear air-guiding channel through the plurality of rear main air-introducing channels.

### CROSS-REFERENCE TO RELATED PATENT APPLICATION

This application claims the benefit of priority to Taiwan Patent Application No. 110133990, filed on Sep. 13, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizer assembly and an airflow-guiding component thereof, and more particularly to a nebulizer assembly and an airflow-guiding component thereof for atomizing a liquid medicine into aerosol particles.

### BACKGROUND OF THE DISCLOSURE

In the related art, an atomization method is often used to treat respiratory diseases. A nebulizer can be used in the atomization method to spray a liquid medicine in the form of aerosol particles that each has a particle size (i.e., diameter) of about 3 to 5 μm, so that the aerosol particles can reach the bronchus and lungs for therapy. The procedure typically involves inhaling the aerosol particles from the liquid medicine through mouths and noses of patients to enter the bronchus, and the particles are then spread to the whole alveolus so that the liquid medicine can be sufficiently absorbed by the human body. This method of delivery is more direct and efficient than oral administration. Currently, the atomization method adapted by the nebulizer generally includes use of a pneumatic atomization module, or an ultrasonic atomization module (such as using a nozzle piece having a plurality of holes), configured for an operating mechanism of the nebulizer. However, there is still room for improvement in the related art for use of the nebulizer.

### SUMMARY OF THE DISCLOSURE

In response to the above-referenced technical inadequacy, the present disclosure provides a nebulizer assembly and an airflow-guiding component thereof to cause an aerosol stream of liquid medicine to be moved forward through external airflow introduced by a plurality of rear main air-introducing channels.

One aspect of the present disclosure provides an airflow-guiding component, which includes an air-guiding part and a structural matching part. The air-guiding part includes a first hollow tube body, a second hollow tube body, and an air-guiding structure connected between the first hollow tube body and the second hollow tube body. The structural matching part is connected to the first hollow tube body of the air-guiding part. The air-guiding structure has a plurality of rear main air-introducing channels surrounding the first hollow tube body, and each of the rear main air-introducing channels has a rear main air-introducing opening perpendicular or inclined to the structural matching part.

Another aspect of the present disclosure provides a nebulizer assembly, which includes a nebulizer host and an airflow-guiding component. The nebulizer host includes a host main body, a medicine-containing module disposed on the host main body, and a nebulizer module disposed inside the medicine-containing module. The airflow-guiding component is assembled on the nebulizer host. The airflow-guiding component includes an air-guiding part and a structural matching part. The air-guiding part includes a first hollow tube body, a second hollow tube body, and an air-guiding structure connected between the first hollow tube body and the second hollow tube body. The structural matching part is connected to the first hollow tube body of the air-guiding part. The air-guiding structure has a plurality of rear main air-introducing channels surrounding the first hollow tube body, and each of the rear main air-introducing channels has a rear main air-introducing opening perpendicular or inclined to the structural matching part.

In some embodiments, the rear main air-introducing opening of each of the rear main air-introducing channels is inclined relative to the first hollow tube body or the structural matching part at an angle ranging from 80 to 100 degrees. The structural matching part has a matching portion disposed on an outer peripheral surface thereof and configured for matching with a matching portion of a nebulizer host. The airflow-guiding component is assembled on the nebulizer host by matching together the matching portion of the airflow-guiding component and the matching portion of the nebulizer host, and the rear main air-introducing opening of each of the rear main air-introducing channels is configured to face the nebulizer host. The structural matching part has a front air-guiding channel formed therein, and the front air-guiding channel is configured to be communicated with a nebulizer module of the nebulizer host. The air-guiding part has a rear air-guiding channel formed therein, and the rear air-guiding channel is communicated with the front air-guiding channel. The rear main air-introducing channels are communicated with the rear air-guiding channel, and external air outside the airflow-guiding component is guided into the rear air-guiding channel through the rear main air-introducing channels. When aerosol streams generated by the nebulizer module are transferred to the rear air-guiding channel, external air is guided into the rear air-guiding channel through the rear main air-introducing channels, and a plurality of medicinal particles of the aerosol streams are moved forward following external air that has been guided into the rear air-guiding channel through the rear main air-introducing channels.

In some embodiments, the air-guiding structure includes a plurality of front main air-guiding walls and a rear main air-guiding wall, the front main air-guiding walls are sequentially connected and surroundingly disposed on the first hollow tube body, and the rear main air-guiding wall is surroundingly connected between the plurality of front main air-guiding walls and the second hollow tube body. Each of the rear main air-introducing channels is formed between a corresponding one of the plurality of front main air-guiding walls and the first hollow tube body, and the rear main air-introducing opening of each of the rear main air-introducing channels is perpendicular or inclined to the first hollow tube body. The air-guiding structure includes at least one lateral auxiliary air-guiding wall, and the at least one lateral auxiliary air-guiding wall is convexly disposed on the rear main air-guiding wall. The at least one lateral auxiliary air-guiding wall has a rear auxiliary air-introducing channel, and the rear auxiliary air-introducing channel has a rear auxiliary air-introducing opening perpendicular or inclined to the rear main air-guiding wall. The front main air-guiding walls are sequentially connected to form an outer peripheral surface with a circular or a polygonal profile, a nozzle opening of the second hollow tube body is shaped as a circle or an ellipse, and a radial distance of the rear main air-guiding wall is gradually decreased from the first hollow tube body to the second hollow tube body.

In some embodiments, the air-guiding structure includes a plurality of front main air-guiding walls, a plurality of rear main air-guiding walls, and a plurality of first connection walls, the plurality of front main air-guiding walls are separate from each other and surroundingly disposed on the first hollow tube body, each of the plurality of rear main air-guiding walls is connected between a corresponding one of the plurality of front main air-guiding walls and the second hollow tube body, and each of the first connection walls is connected between the first hollow tube body and the second hollow tube body, and is connected between two adjacent ones of the plurality of rear main air-guiding walls. Each of the rear main air-introducing channels is formed between a corresponding one of the plurality of front main air-guiding walls and the first hollow tube body, and the rear main air-introducing opening of each of the plurality of rear main air-introducing channels is perpendicular or inclined to the first hollow tube body.

In some embodiments, the air-guiding structure includes a plurality of rear main air-guiding walls, and a plurality of first connection walls, each of the rear main air-guiding walls is connected between the first hollow tube body and the second hollow tube body, and each of the plurality of first connection walls is connected between the first hollow tube body and the second hollow tube body, and is connected between two adjacent ones of the plurality of rear main air-guiding walls. The air-guiding part has a rear air-guiding channel formed therein, each of the plurality of rear main air-introducing channels is formed between a corresponding one of the plurality of rear main air-guiding walls and the rear air-guiding channel, and the rear main air-introducing opening of each of the plurality of rear main air-introducing channels is perpendicular or inclined to the first hollow tube body. The first hollow tube body has a plurality of lateral main air-introducing openings separate from each other, each of the plurality of lateral main air-introducing openings extends along an outer peripheral surface of the first hollow tube body, and the lateral main air-introducing openings are respectively communicated with the rear main air-introducing openings. The air-guiding structure includes a plurality of second connection walls separate from each other, and each of the plurality of second connection walls is connected between two adjacent ones of the plurality of rear main air-guiding walls, and is adjacent to a corresponding one of the first connection walls. The air-guiding structure includes a surrounding extending wall surrounding the plurality of lateral main air-introducing openings, and the surrounding extending wall has a plurality of lateral auxiliary air-introducing openings respectively corresponding to the lateral main air-introducing openings.

Therefore, in the nebulizer assembly and the airflow-guiding component thereof provided by the present disclosure, external air outside the airflow-guiding component is guided into the rear air-guiding channel through the rear main air-introducing channels by the following means: “the air-guiding part including a first hollow tube body, a second hollow tube body, and an air-guiding structure connected between the first hollow tube body and the second hollow tube body”, “the structural matching part being connected to the first hollow tube body,” and “the air-guiding structure having a plurality of rear main air-introducing channels surrounding the first hollow tube body, and each of the rear main air-introducing channels having a rear main air-introducing opening perpendicular or inclined to the structural matching part.” Whereby, when aerosol streams generated by the nebulizer module are transferred to the rear air-guiding channel, external air can be guided into the rear air-guiding channel through the rear main air-introducing channels of the air-guiding part, and a plurality of medicinal particles of the aerosol streams can be moved forward following external air that has been guided into the rear air-guiding channel through the rear main air-introducing channels.

These and other aspects of the present disclosure will become apparent from the following description of the embodiment taken in conjunction with the following drawings and their captions, and variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective exploded view of a nebulizer assembly according to a first embodiment of the present disclosure;

FIG. 2 is a partial schematic cross-sectional view of the nebulizer assembly according to the first embodiment of the present disclosure;

FIG. 3 is a schematic perspective view of an airflow-guiding component according to the first embodiment of the present disclosure;

FIG. 4 is a cross-sectional view taken along line IV-IV of FIG. 3;

FIG. 5 is a schematic perspective view of the airflow-guiding component according to a second embodiment of the present disclosure;

FIG. 6 is a cross-sectional view taken along line VI-VI of FIG. 5;

FIG. 7 is a schematic perspective view of the airflow-guiding component according to a third embodiment of the present disclosure;

FIG. 8 is a cross-sectional view taken along line VIII-VIII of FIG. 7;

FIG. 9 is a schematic perspective view of the airflow-guiding component according to a fourth embodiment of the present disclosure;

FIG. 10 is a cross-sectional view taken along line X-X of FIG. 9;

FIG. 11 is a schematic perspective view of the airflow-guiding component according to a fifth embodiment of the present disclosure;

FIG. 12 is a cross-sectional view taken along line XII-XII of FIG. 11;

FIG. 13 is a schematic perspective view of the airflow-guiding component according to a sixth embodiment of the present disclosure;

FIG. 14 is a cross-sectional view taken along line XIV-XIV of FIG. 13;

FIG. 15 is a schematic perspective view of the airflow-guiding component according to a seventh embodiment of the present disclosure; and

FIG. 16 is a cross-sectional view taken along line XVI-XVI of FIG. 15.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

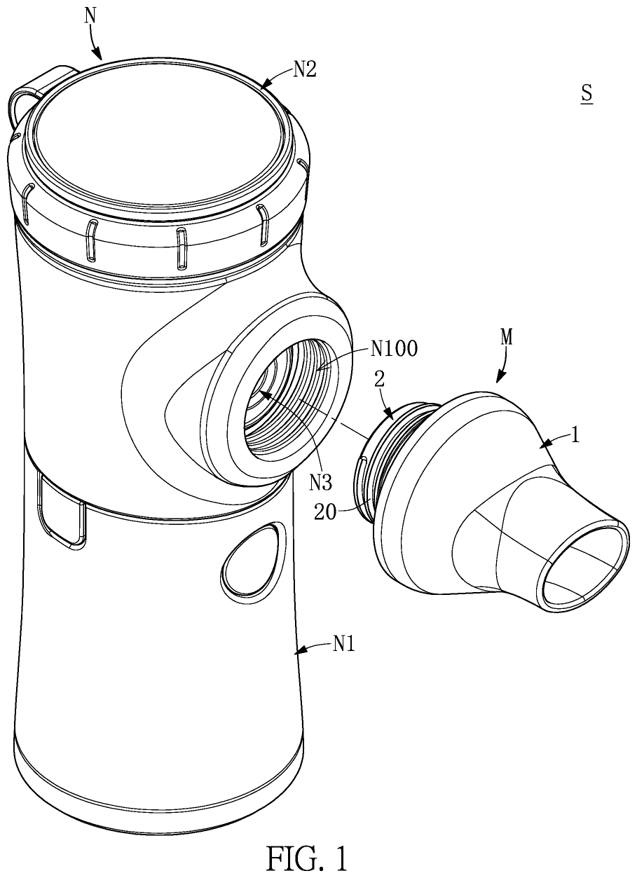

Referring to FIG. 1 to FIG. 16, the present disclosure provides a nebulizer assembly S, and the nebulizer assembly S includes a nebulizer host N and an airflow-guiding component M (such as a mouthpiece nozzle or a spray nozzle). The nebulizer host N includes a host main body N1, a medicine-containing module N2 (such as a medicine reservoir, or a medicine cup) disposed on the host main body N1, and a nebulizer module N3 disposed inside the medicine-containing module N2, and the airflow-guiding component M includes an air-guiding part 1 and a structural matching part 2. More particularly, the air-guiding part 1 includes a first hollow tube body 11, a second hollow tube body 12, and an air-guiding structure 13 connected between the first hollow tube body 11 and the second hollow tube body 12. The structural matching part 2 is connected to the first hollow tube body 11 of the air-guiding part 1. In addition, the air-guiding structure 13 has a plurality of rear main air-introducing channels 1300 surrounding the first hollow tube body 11, and each of the rear main air-introducing channels 1300 has a rear main air-introducing opening 13000 perpendicular or inclined to the structural matching part 2.

Therefore, when the nebulizer host N is turned on for using the nebulizer module N3 of the nebulizer host N to generate aerosol streams F, external air A outside the airflow-guiding component M can be guided into the rear air-guiding channel 1000 of the air-guiding part 1 through the plurality of rear main air-introducing channels 1300, so that a plurality of medicinal particles (i.e., aerosol particles) of the aerosol streams F can be moved forward following (or by) external air A guided into the rear air-guiding channel 1000 through the rear main air-introducing channels 1300.

### First Embodiment

Referring to FIG. 1 to FIG. 4, a first embodiment of the present disclosure provides an airflow-guiding component M, and a nebulizer assembly S using the airflow-guiding component M.

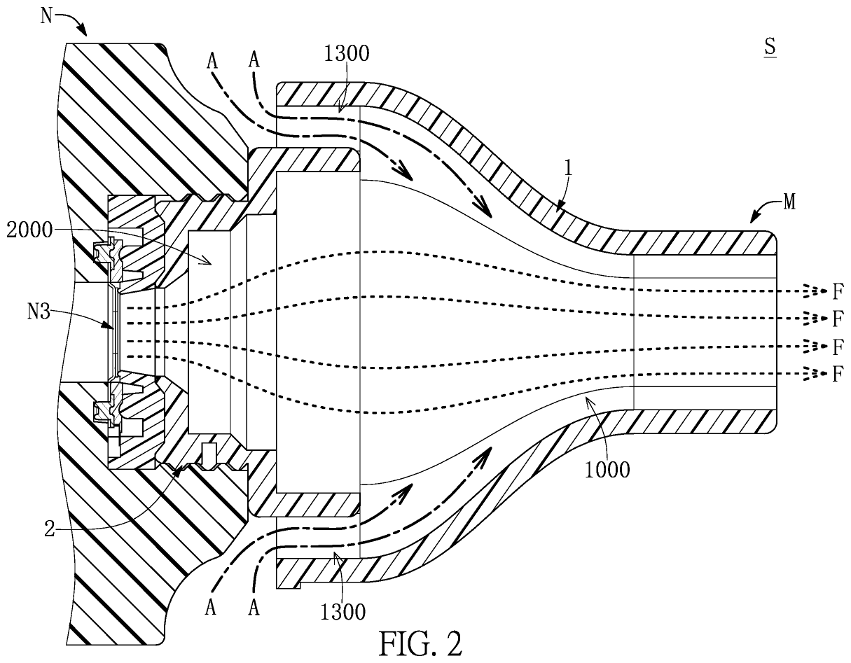

Firstly, referring to FIG. 1 and FIG. 2, the nebulizer assembly S includes a nebulizer host N and an airflow-guiding component M assembled on the nebulizer host N. The nebulizer host N includes a host main body N1, a medicine-containing module N2 disposed on the host main body N1, and a nebulizer module N3 disposed inside the medicine-containing module N2, and the airflow-guiding component M includes an air-guiding part 1 and a structural matching part 2. For example, the medicine-containing module N2 can be detachably or fixedly disposed on the host main body N1 in order to receive a predetermined liquid medicine, and the nebulizer module N3 can be configured for transforming the predetermined liquid medicine into aerosol streams F by a nozzle piece having a plurality of holes. In addition, a matching portion is disposed on an outer peripheral surface of the structural matching part 2 and configured for matching with a matching portion of a nebulizer host N, and the airflow-guiding component M can be assembled on the nebulizer host N by matching together the matching portion of the airflow-guiding component M and the matching portion of the nebulizer host N. For example, the matching portions can be illustrated as a thread structure 20 disposed on an outer peripheral surface of the structural matching part 2 and a thread structure N100 of a nebulizer host N, configured for matching with each other, and the airflow-guiding component M can be assembled on the nebulizer host N by matching the thread structure 20 of the airflow-guiding component M and the thread structure N100 of the nebulizer host N. Hence, the airflow-guiding component M can be replaceably assembled on the nebulizer host N according to different requirements. However, the aforementioned is merely an example, and is not intended to limit the scope of the present disclosure.

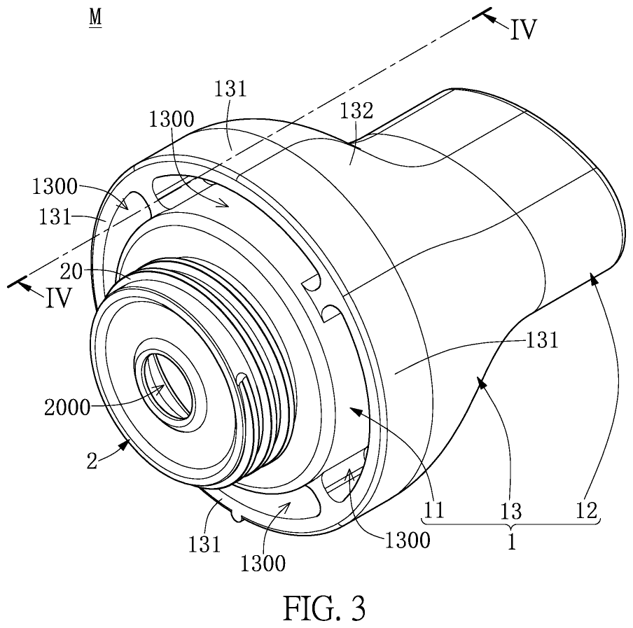

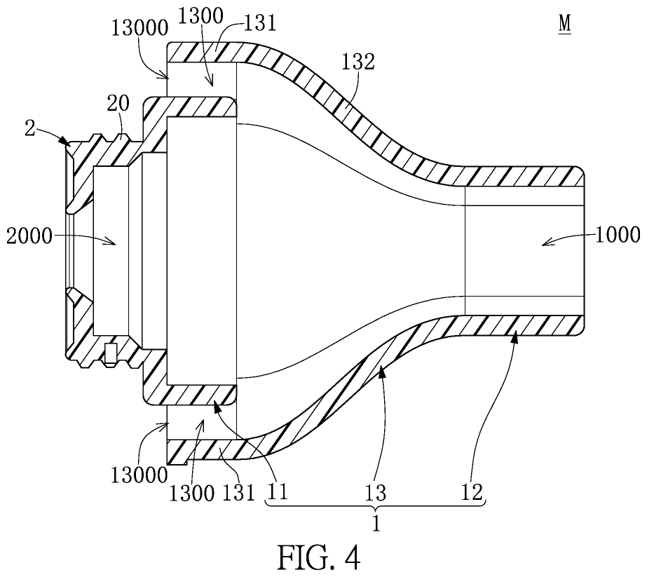

Moreover, referring to FIG. 3 and FIG. 4, the air-guiding part 1 includes a first hollow tube body 11, a second hollow tube body 12, and an air-guiding structure 13 connected between the first hollow tube body 11 and the second hollow tube body 12. In addition, the structural matching part 2 is connected to the first hollow tube body 11 of the air-guiding part 1 so as to match with the thread structure N100 of the nebulizer host N (as shown in FIG. 1). More particularly, the air-guiding structure 13 includes a plurality of front main air-guiding walls 131 and a rear main air-guiding wall 132. The front main air-guiding walls 131 are sequentially connected and surroundingly disposed on the first hollow tube body 11, and the rear main air-guiding wall 132 is surroundingly connected between the front main air-guiding walls 131 and the second hollow tube body 12. In other words, the air-guiding structure 13 includes a third hollow tube body (not labeled) extended from the first hollow tube body 11 to the second hollow tube body 12, and the third hollow tube body includes a plurality of support posts (not labeled) surroundingly connected to the first hollow tube body 11 so as to form a plurality of through channels (not labeled) between the third hollow tube body and the first hollow tube body 11. In addition, a radical distance of the rear main air-guiding wall 132 is gradually decreased from the first hollow tube body 11 to the second hollow tube body 12 (i.e., the rear main air-guiding wall 132 is shown as a tapered tubular structure). For example, the plurality of front main air-guiding wall 131 can be sequentially connected to form an outer peripheral surface with a circular profile or any profile in any kind of shape, and a mouthpiece opening of the second hollow tube body 12 can be shaped as a circle, an ellipse or any kind of shape. However, the aforementioned is merely an example, and is not intended to limit the scope of the present disclosure.

It should be noted that, referring to FIG. 3 and FIG. 4, the air-guiding structure 13 has a plurality of rear main air-introducing channels 1300 (i.e., main rear intake passages) surrounding the first hollow tube body 11, and each one of the plurality of rear main air-introducing channels 1300 has a rear main air-introducing opening 13000 (i.e., a main rear inlet) perpendicular or inclined to the structural matching part 2. In addition, when the airflow-guiding component M is assembled on the nebulizer host N by matching the thread structure 20 of the airflow-guiding component M with the thread structure N100 of the nebulizer host N (as shown in FIG. 1), the rear main air-introducing opening 13000 of each of the plurality of rear main air-introducing channels 1300 is faced to the nebulizer host N (as shown in FIG. 2), so that the rear main air-introducing opening 13000 is functioned as a rear inlet, not a lateral inlet. More particularly, each of the plurality of rear main air-introducing channels 1300 is formed between a corresponding one of the front main air-guiding walls 131, and the first hollow tube body 11 (or each of the plurality of rear main air-introducing channels 1300 is formed between two adjacent ones of the support posts), and the rear main air-introducing opening 13000 of each of the plurality of rear main air-introducing channels 1300 can be perpendicular or inclined to the first hollow tube body 11. For example, the rear main air-introducing opening 13000 of each of the plurality of rear main air-introducing channels 1300 can be inclined relative to the first hollow tube body 11 (such as an outer surface of the first hollow tube body 11) or the structural matching part 2 (such as an outer surface of the structural matching part 2) at a predetermined angle ranging from 80 to 100 degrees (such as an arbitrary positive integer between 80 and 100 degrees). However, the aforementioned is merely an example, and is not intended to limit the scope of the present disclosure.

Furthermore, referring to FIG. 2 to FIG. 4, the structural matching part 2 has a front air-guiding channel 2000 formed therein, and the front air-guiding channel 2000 of the structural matching part 2 can be configured to communicate with a nebulizer module N3 of the nebulizer host N. In addition, the air-guiding part 1 has a rear air-guiding channel 1000 formed therein, and the rear air-guiding channel 1000 of the air-guiding part 1 is communicated with the front air-guiding channel 2000 of the structural matching part 2. More particularly, the plurality of rear main air-introducing channels 1300 are communicated with the rear air-guiding channel 1000 of the air-guiding part 1, so that external air A (or ambient air) outside the airflow-guiding component M can be guided or introduced into the rear air-guiding channel 1000 through the plurality of rear main air-introducing channels 1300 (as shown in FIG. 2).

Therefore, as shown in FIG. 2, when aerosol streams F (or atomized streams) generated by the nebulizer module N3 are transferred to the rear air-guiding channel 1000 of the air-guiding part 1 (i.e., when the airflow-guiding component M is used to perform an inhalation action), external air A can be guided or introduced into the rear air-guiding channel 1000 through the plurality of rear main air-introducing channels 1300, so that a plurality of medicinal particles of the aerosol streams F can be moved forward following (or by) external air A that has been guided or introduced into the rear air-guiding channel 1000 through the plurality of rear main air-introducing channels 1300. Hence, when aerosol streams F having the medicinal particles are generated by the nebulizer module N3, external air A is guided or introduced into the rear air-guiding channel 1000 through the plurality of rear main air-introducing channels 1300, so that a forward flow velocity of aerosol streams F can be increased and a flow collision rate (or a large drop formation rate due to particle collision) of aerosol streams F can be decreased. Therefore, the nebulizer assembly S of the airflow-guiding component M can be used to decrease a residual drug dosage (i.e., a formation rate of a large drop of the medicinal particles which is dropped inside the airflow-guiding component M) and increase a spray-out rate of particles (i.e., a rate of the medicinal particles of aerosol streams F that are sprayed out from the airflow-guiding component M).

### Second Embodiment

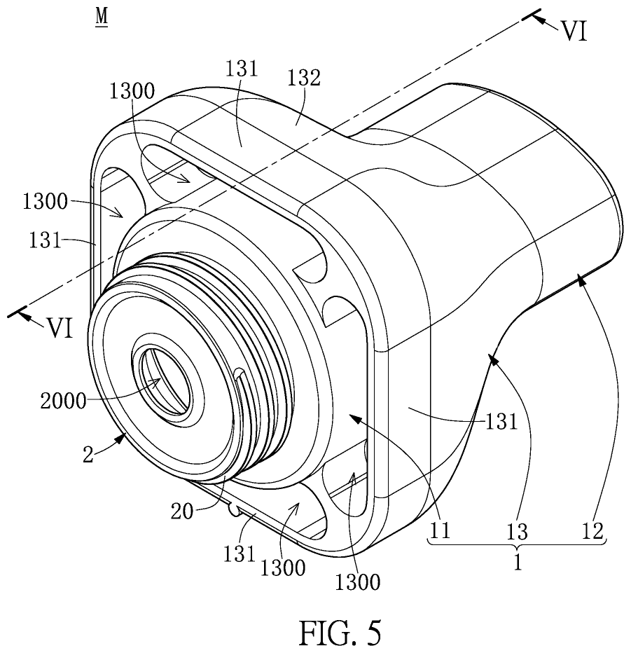

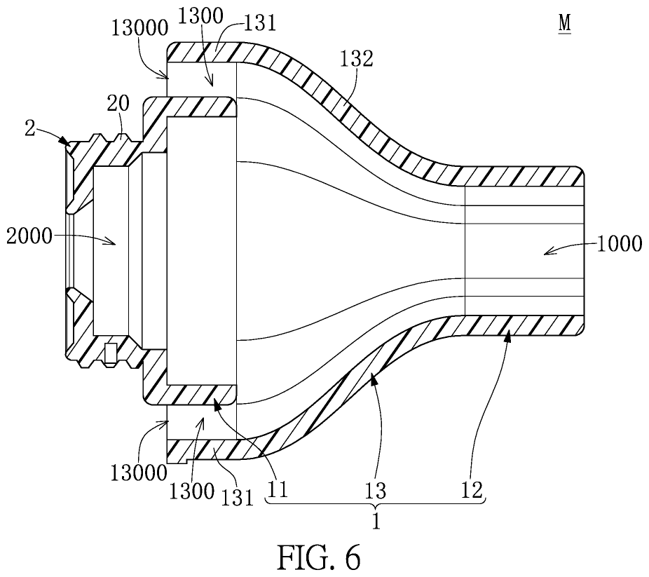

Referring to FIG. 5 and FIG. 6, a second embodiment of the present disclosure provides an airflow-guiding component M, and the airflow-guiding component M can be assembled on a nebulizer assembly (not shown in figures) in the same way shown in the first embodiment of FIG. 1. As shown in FIG. 5 and FIG. 6, compared to FIG. 3 and FIG. 4, the major difference between the second embodiment and the first embodiment is shown as the following: in the second embodiment, the plurality of front main air-guiding wall 131 can be sequentially connected to form an outer peripheral surface with a polygonal profile. According to different requirements, the front main air-guiding wall 131 can be sequentially connected to form an outer peripheral surface with a circular profile (as shown in the first embodiment), with a polygonal profile (as shown in the second embodiment) or with a profile in any kind of shape. However, the aforementioned is merely an example, and is not intended to limit the scope of the present disclosure.

### Third Embodiment

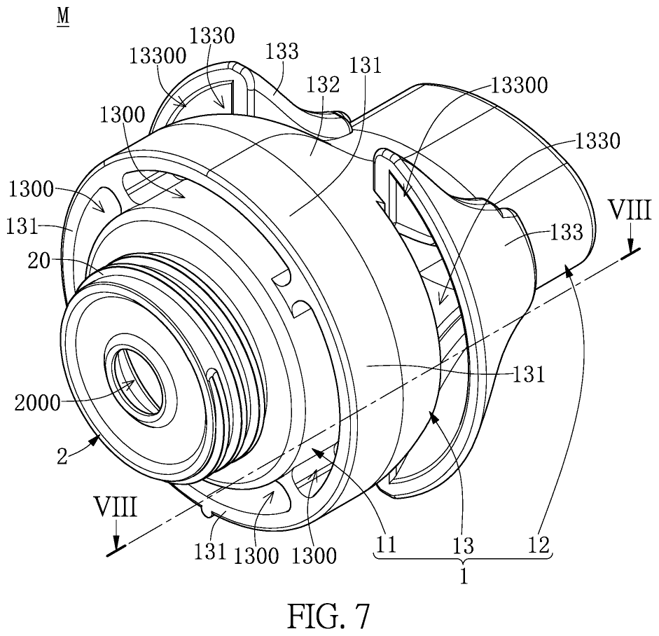

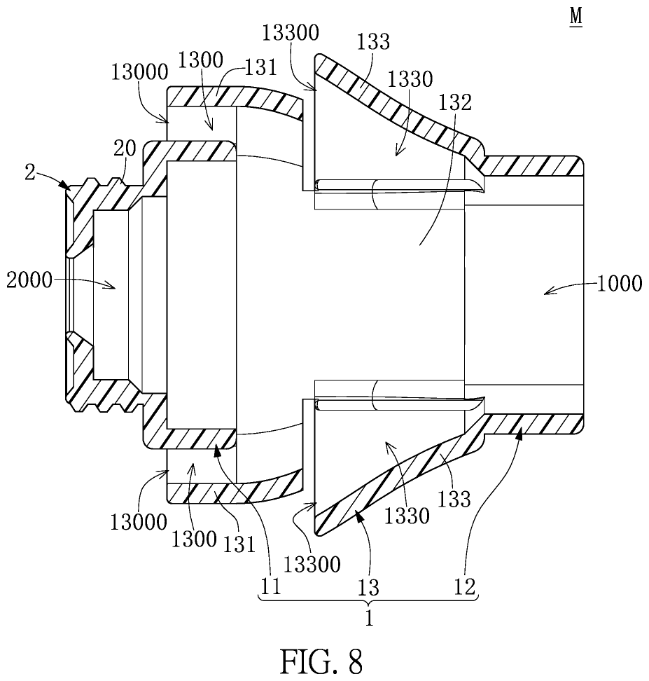

Referring to FIG. 7 and FIG. 8, a third embodiment of the present disclosure provides an airflow-guiding component M, and the airflow-guiding component M can be assembled on a nebulizer assembly (not shown in figures) in the same way as shown in the first embodiment of FIG. 1. As shown in FIG. 7 and FIG. 8, compared to FIG. 3 and FIG. 4, the major difference between the third embodiment and the first embodiment is shown as the following: in the third embodiment, the air-guiding structure 13 includes two lateral auxiliary air-guiding walls 133 (or at least one lateral auxiliary air-guiding wall 133) symmetrical to each other, and the lateral auxiliary air-guiding wall 133 is convexly disposed on the rear main air-guiding wall 132.

More particularly, referring to FIG. 7 and FIG. 8, the lateral auxiliary air-guiding wall 133 has a rear auxiliary air-introducing channel 1330, and the rear auxiliary air-introducing channel 1330 has a rear auxiliary air-introducing opening 13300 perpendicular or inclined to the rear main air-guiding wall 132. For example, the rear auxiliary air-introducing opening 13300 of each of the rear auxiliary air-introducing channels 1330 is inclined relative to an axis line of the airflow-guiding component M at a predetermined angle ranging from 80 to 100 degrees (such as an arbitrary positive integer between 80 and 100 degrees). However, the aforementioned is merely an example, and is not intended to limit the scope of the present disclosure.

### Fourth Embodiment

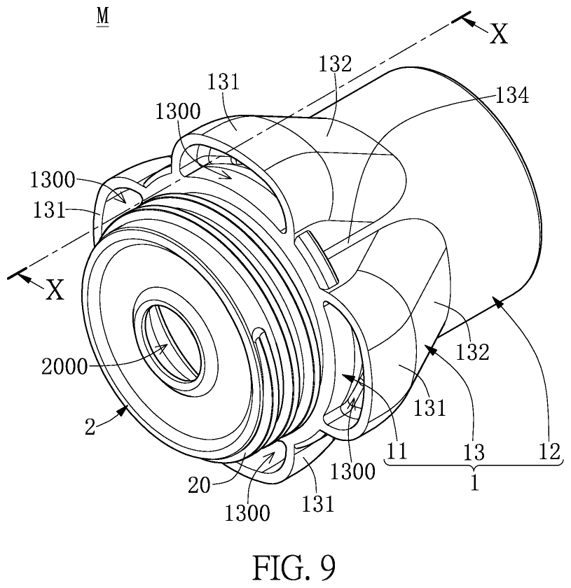

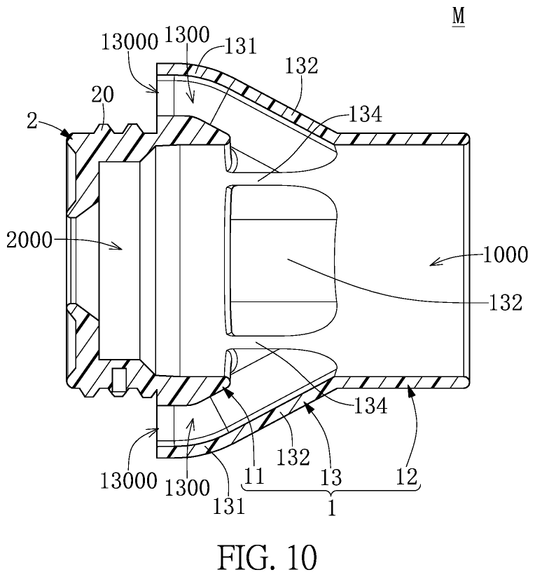

Referring to FIG. 9 and FIG. 10, a fourth embodiment of the present disclosure provides an airflow-guiding component M, and the airflow-guiding component M can be assembled on a nebulizer assembly (not shown in figures) in the same way as shown in the first embodiment of FIG. 1. As shown in FIG. 9 and FIG. 10, compared to FIG. 3 and FIG. 4, the major difference between the fourth embodiment and the first embodiment is shown as the following: in the fourth embodiment, the air-guiding structure 13 includes a plurality of front main air-guiding walls 131, a plurality of rear main air-guiding walls 132, and a plurality of first connection walls 134.

More particularly, referring to FIG. 9 and FIG. 10, the plurality of front main air-guiding walls 131 are separate from each other and surroundingly disposed on the first hollow tube body 11, each of the plurality of rear main air-guiding walls 132 is connected between a corresponding one of the plurality of front main air-guiding walls 131, and the second hollow tube body 12, and each of the plurality of first connection walls 134 is connected between the first hollow tube body 11 and the second hollow tube body 12, and is connected between two adjacent ones of the plurality of rear main air-guiding walls 132. In addition, each of the plurality of rear main air-introducing channels 1300 is formed between a corresponding one of the plurality of front main air-guiding walls 131, and the first hollow tube body 11, and the rear main air-introducing opening 13000 of each of the plurality of rear main air-introducing channels 1300 can be perpendicular or inclined to the first hollow tube body 11.

### Fifth Embodiment

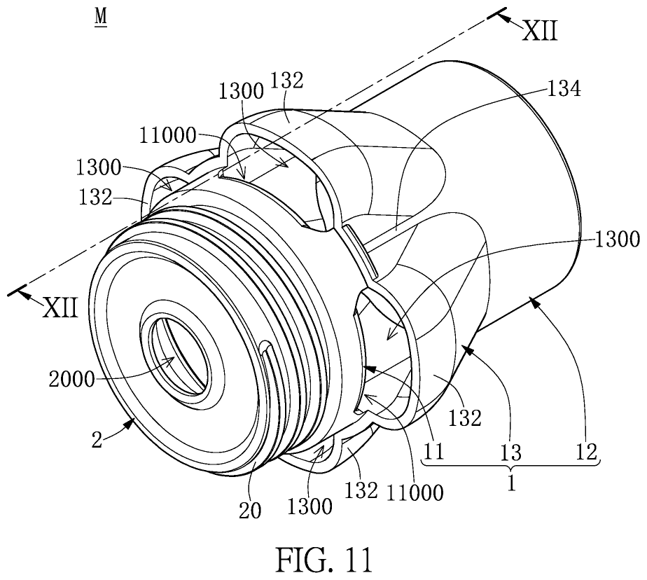

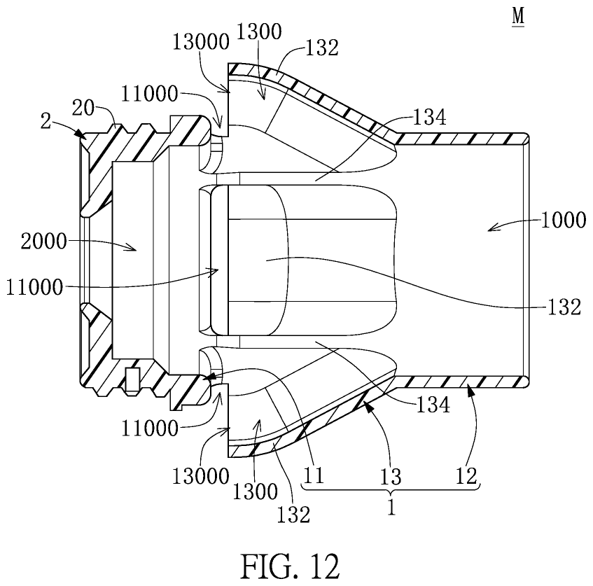

Referring to FIG. 11 and FIG. 12, a fifth embodiment of the present disclosure provides an airflow-guiding component M, and the airflow-guiding component M can be assembled on a nebulizer assembly (not shown in figures) in the same way as shown in the first embodiment of FIG. 1. As shown in FIG. 11 and FIG. 12, compared to FIG. 9 and FIG. 10, the major difference between the fifth embodiment and the fourth embodiment is shown as the following: in the fifth embodiment, the air-guiding structure 13 includes a plurality of rear main air-guiding walls 132, and a plurality of first connection walls 134.

More particularly, referring to FIG. 11 and FIG. 12, each of the plurality of rear main air-guiding walls 132 is connected between the first hollow tube body 11 and the second hollow tube body 12, and the first hollow tube body 11 is not surrounded by the rear main air-guiding walls 132. Each of the plurality of first connection walls 134 is connected between the first hollow tube body 11 and the second hollow tube body 12, and is connected between two adjacent ones of the plurality of rear main air-guiding walls 132. In addition, the air-guiding part 1 has a rear air-guiding channel 1000 formed therein, each of the plurality of rear main air-introducing channels 1300 is formed between a corresponding one of the plurality of rear main air-guiding walls 132 and the rear air-guiding channel 1000, and the rear main air-introducing opening 13000 of each of the plurality of rear main air-introducing channels 1300 can be perpendicular or inclined to the first hollow tube body 11 or an axis line of the airflow-guiding component M.

It should be noted that, referring to FIG. 11 and FIG. 12, the first hollow tube body 11 has a plurality of lateral main air-introducing openings 11000 separate from each other, each of the lateral main air-introducing openings 11000 extends along an outer peripheral surface of the first hollow tube body 11, and the plurality of lateral main air-introducing openings 11000 are respectively communicated with the rear main air-introducing openings 13000.

### Sixth Embodiment

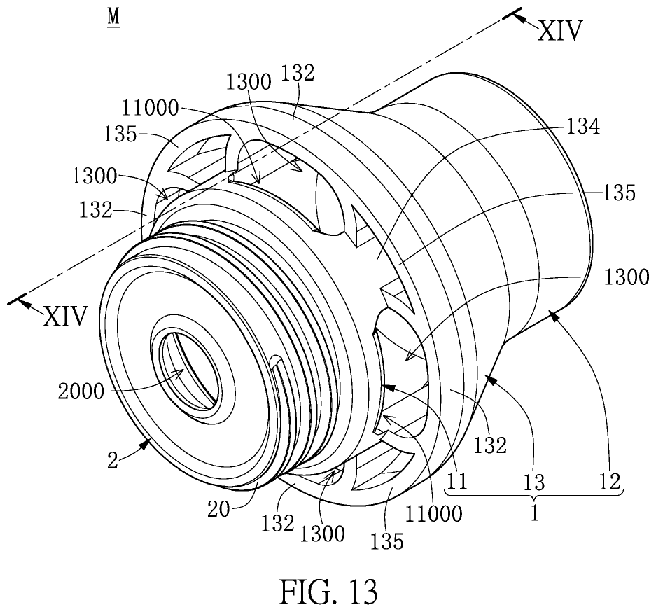

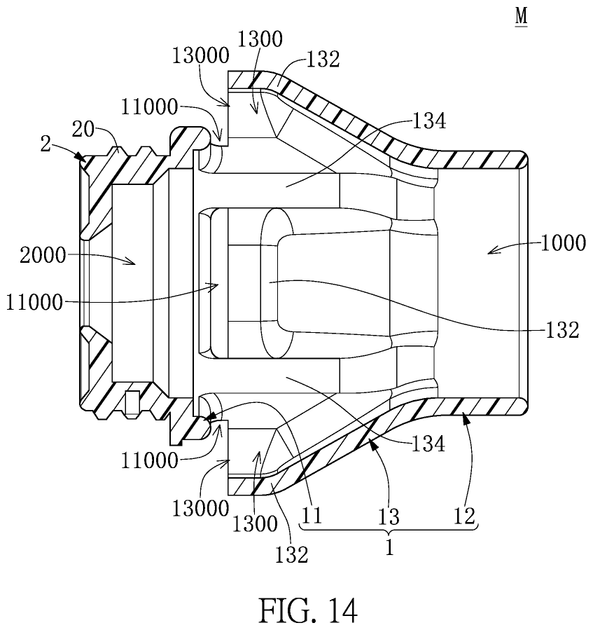

Referring to FIG. 13 and FIG. 14, a sixth embodiment of the present disclosure provides an airflow-guiding component M, and the airflow-guiding component M can be assembled on a nebulizer assembly (not shown in figures) in the same way as shown in the first embodiment of FIG. 1. As shown in FIG. 13 and FIG. 14, compared to FIG. 11 and FIG. 12, the major difference between the sixth embodiment and the fifth embodiment is shown as the following: in the sixth embodiment, the air-guiding structure 13 includes a plurality of second connection walls 135 separate from each other, and each of the plurality of second connection walls 135 is connected between two adjacent ones of the plurality of rear main air-guiding walls 132, and is adjacent to a corresponding one of the plurality of first connection walls 134 to increase integrally structural strength of the air-guiding structure 13.

### Seventh Embodiment

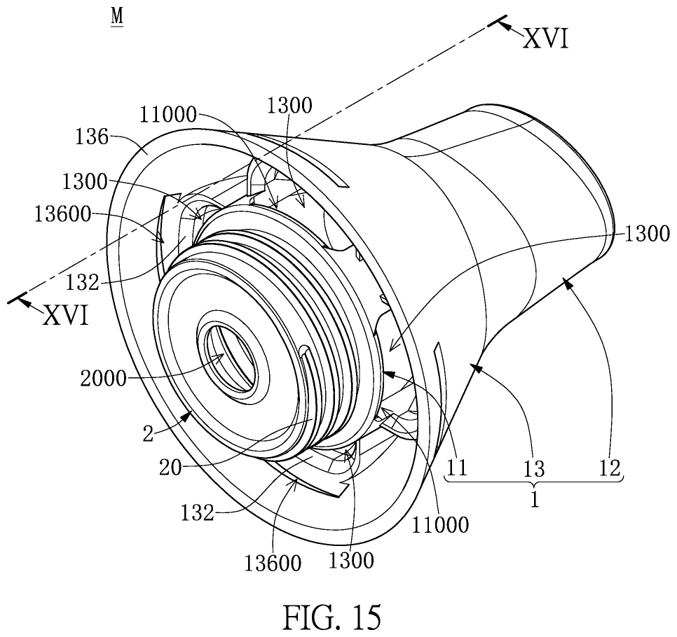

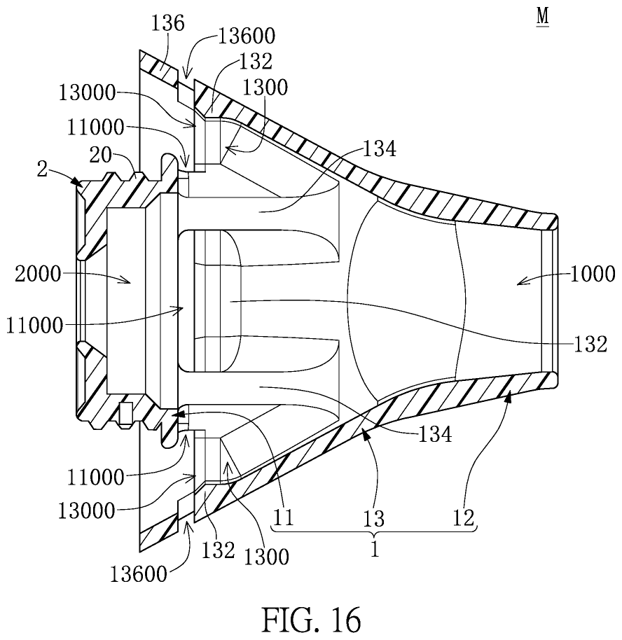

Referring to FIG. 15 and FIG. 16, a seventh embodiment of the present disclosure provides an airflow-guiding component M, and the airflow-guiding component M can be assembled on a nebulizer assembly (not shown in figures) in the same way as shown in the first embodiment of FIG. 1. As shown in FIG. 15 and FIG. 16, compared to FIG. 13 and FIG. 14, the major difference between the seventh embodiment and the sixth embodiment is shown as the following: in the seventh embodiment, the air-guiding structure 13 includes a surrounding extending wall 136 surrounding the plurality of lateral main air-introducing openings 11000, and the surrounding extending wall 136 has a plurality of lateral auxiliary air-introducing openings 13600 respectively corresponding to the plurality of lateral main air-introducing openings 11000. Therefore, after external air (or ambient air) passes through lateral auxiliary air-introducing openings 13600, external air can be guided or introduced into the rear air-guiding channel 1000 through the plurality of lateral main air-introducing openings 11000 and the plurality of rear main air-introducing openings 13000.

### Effectiveness of the Embodiments

In conclusion, in the nebulizer assembly S and the airflow-guiding component M thereof provided by the present disclosure, external air A outside of the airflow-guiding component M can be guided into the rear air-guiding channel 1000 through the rear main air-introducing channels 1300 by the following means: “the air-guiding part 1 including a first hollow tube body 11, a second hollow tube body 12, and an air-guiding structure 13 connected between the first hollow tube body 11 and the second hollow tube body 12”, “the structural matching part 2 being connected to the first hollow tube body 11,” and “the air-guiding structure 13 having a plurality of rear main air-introducing channels 1300 surrounding the first hollow tube body 11, and each of the rear main air-introducing channels 1300 having a rear main air-introducing opening 13000 perpendicular or inclined to the structural matching part 2.”

Whereby, when aerosol streams F generated by the nebulizer module N3 are transferred to the rear air-guiding channel 1000 of the air-guiding part 1, external air A can be guided into the rear air-guiding channel 1000 through the rear main air-introducing channels 1300, and a plurality of medicinal particles of the aerosol streams F can be moved forward following (or by) external air A that has been guided into the rear air-guiding channel 1000 through the rear main air-introducing channels 1300.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An airflow-guiding component, comprising:
an air-guiding part including a first hollow tube body, a second hollow tube body, and an air-guiding structure connected between the first hollow tube body and the second hollow tube body; and
a structural matching part connected to the first hollow tube body;
wherein the air-guiding structure has a plurality of rear main air-introducing channels surrounding the first hollow tube body, and each of the rear main air-introducing channels has a rear main air-introducing opening perpendicular or inclined to the structural matching part.

2. The airflow-guiding component according to claim 1,
wherein the rear main air-introducing opening of each of the plurality of rear main air-introducing channels is inclined relative to the first hollow tube body or the structural matching part at an angle ranging from 80 to 100 degrees;
wherein the structural matching part includes a matching portion disposed on an outer peripheral surface thereof and configured for matching with a matching portion of a nebulizer host, the airflow-guiding component is assembled on the nebulizer host by matching together the matching portion of the airflow-guiding component and the matching portion of the nebulizer host, and the rear main air-introducing opening of each of the plurality of rear main air-introducing channels is configured to face the nebulizer host;
wherein the structural matching part includes a front air-guiding channel therein, and the front air-guiding channel is configured to be communicated with a nebulizer module of the nebulizer host;
wherein the air-guiding part has a rear air-guiding channel formed therein, and the rear air-guiding channel is communicated with the front air-guiding channel;
wherein the plurality of rear main air-introducing channels are communicated with the rear air-guiding channel, external air outside the airflow-guiding component is guided into the rear air-guiding channel through the plurality of rear main air-introducing channels;
wherein, when aerosol streams generated by the nebulizer module are transferred to the rear air-guiding channel, external air is guided into the rear air-guiding channel through the plurality of rear main air-introducing channels, a plurality of medicinal particles of the aerosol streams are moved forward following external air that has been guided into the rear air-guiding channel through the plurality of rear main air-introducing channels.

3. The airflow-guiding component according to claim 1,
wherein the air-guiding structure includes a plurality of front main air-guiding walls and a rear main air-guiding wall, the plurality of front main air-guiding walls are sequentially connected and surroundingly disposed on the first hollow tube body, and the rear main air-guiding wall is surroundingly connected between the plurality of front main air-guiding walls and the second hollow tube body;
wherein each of the plurality of rear main air-introducing channels is formed between a corresponding one of the plurality of front main air-guiding walls and the first hollow tube body, and each of the plurality of rear main air-introducing openings of each of the plurality of rear main air-introducing channels is perpendicular or inclined to the first hollow tube body;
wherein the air-guiding structure includes at least one lateral auxiliary air-guiding wall, and the at least one lateral auxiliary air-guiding wall is convexly disposed on the rear main air-guiding wall;
wherein the at least one lateral auxiliary air-guiding wall has a rear auxiliary air-introducing channel, and the rear auxiliary air-introducing channel has a rear auxiliary air-introducing opening perpendicular or inclined to the rear main air-guiding wall;
wherein the plurality of front main air-guiding walls are sequentially connected to form an outer peripheral surface having a circular or a polygonal surface profile, a nozzle opening of the second hollow tube body is shaped as a circle or an ellipse, and a radial distance of the rear main air-guiding wall is gradually decreased from the first hollow tube body to the second hollow tube body.

4. The airflow-guiding component according to claim 1,
wherein the air-guiding structure includes a plurality of front main air-guiding walls, a plurality of rear main air-guiding walls, and a plurality of first connection walls, the plurality of front main air-guiding walls are separate from each other and surroundingly disposed on the first hollow tube body, each of the plurality of rear main air-guiding walls is connected between a corresponding one of the plurality of front main air-guiding walls and the second hollow tube body, and each of the plurality of first connection walls is connected between the first hollow tube body and the second hollow tube body, and is connected between two adjacent ones of the plurality of rear main air-guiding walls;
wherein each of the plurality of rear main air-introducing channels is formed between a corresponding one of the plurality of front main air-guiding walls and the first hollow tube body, and each one of the plurality of rear main air-introducing openings of each of the plurality of rear main air-introducing channels is perpendicular or inclined to the first hollow tube body.

5. The airflow-guiding component according to claim 1,
wherein the air-guiding structure includes a plurality of rear main air-guiding walls and a plurality of first connection walls, each of the plurality of rear main air-guiding walls is connected between the first hollow tube body and the second hollow tube body, and each of the plurality of first connection walls is connected between the first hollow tube body and the second hollow tube body, and is connected between two adjacent ones of the plurality of rear main air-guiding walls;
wherein the air-guiding part has a rear air-guiding channel formed therein, each of the plurality of rear main air-introducing channels is formed between one of the plurality of rear main air-guiding walls and one of the plurality of rear air-guiding channel corresponding to each other, and each of the plurality of rear main air-introducing openings of each of the rear main air-introducing channels is perpendicular or inclined to the first hollow tube body;
wherein the first hollow tube body has a plurality of lateral main air-introducing openings separate from each other, each of the plurality of lateral main air-introducing openings extends along an outer peripheral surface of the first hollow tube body, and the plurality of lateral main air-introducing openings are respectively communicated with the plurality of rear main air-introducing openings;
wherein the air-guiding structure includes a plurality of second connection walls separate from each other, and each of the plurality of second connection walls is connected between two adjacent ones of the plurality of rear main air-guiding walls, and is adjacent to a corresponding one of the plurality of first connection walls;
wherein the air-guiding structure includes a surrounding extending wall surrounding the plurality of lateral main air-introducing openings, and the surrounding extending wall has a plurality of lateral auxiliary air-introducing openings respectively corresponding to the plurality of lateral main air-introducing openings.

6. A nebulizer assembly, comprising:
a nebulizer host including a host main body, a medicine-containing module disposed on the host main body, and a nebulizer module disposed inside the medicine-containing module; and
an airflow-guiding component assembled on the nebulizer host;
wherein the airflow-guiding component includes:
an air-guiding part including a first hollow tube body, a second hollow tube body, and an air-guiding structure connected between the first hollow tube body and the second hollow tube body; and
a structural matching part connected to the first hollow tube body;
wherein the air-guiding structure has a plurality of rear main air-introducing channels surrounding the first hollow tube body, and each of the rear main air-introducing channels has a rear main air-introducing opening perpendicular or inclined to the structural matching part.

7. The nebulizer assembly according to claim 6,
wherein the rear main air-introducing opening of each of the plurality of rear main air-introducing channels is inclined relative to the first hollow tube body or the structural matching part at an angle ranging from 80 to 100 degrees;
wherein the structural matching part includes a matching portion disposed on an outer peripheral surface thereof and configured for matching with a matching portion of the nebulizer host, the airflow-guiding component is assembled on the nebulizer host by matching together the matching portion of the airflow-guiding component and the matching portion of the nebulizer host, and the rear main air-introducing opening of each of the plurality of rear main air-introducing channels is configured to face the nebulizer host;
wherein the structural matching part includes a front air-guiding channel therein, and the front air-guiding channel is configured to be communicated with the nebulizer module;
wherein the air-guiding part has a rear air-guiding channel formed therein, and the rear air-guiding channel is communicated with the front air-guiding channel;
wherein the plurality of rear main air-introducing channels are communicated with the rear air-guiding channel, external air outside the airflow-guiding component is guided into the rear air-guiding channel through the plurality of rear main air-introducing channels;
wherein, when aerosol streams generated by the nebulizer module are transferred to the rear air-guiding channel, external air is guided into the rear air-guiding channel through the plurality of rear main air-introducing channels, a plurality of medicinal particles of the aerosol streams are moved forward following external air that has been guided into the rear air-guiding channel through the plurality of rear main air-introducing channels.

8. The nebulizer assembly according to claim 6,
wherein the air-guiding structure includes a plurality of front main air-guiding walls and a rear main air-guiding wall, the plurality of front main air-guiding walls are sequentially connected and surroundingly disposed on the first hollow tube body, and the rear main air-guiding wall is surroundingly connected between the plurality of front main air-guiding walls and the second hollow tube body;
wherein each of the plurality of rear main air-introducing channels is formed between a corresponding one of the plurality of front main air-guiding walls and the first hollow tube body, and each of the plurality of rear main air-introducing openings of each of the plurality of rear main air-introducing channels is perpendicular or inclined to the first hollow tube body;
wherein the air-guiding structure includes at least one lateral auxiliary air-guiding wall, and the at least one lateral auxiliary air-guiding wall is convexly disposed on the rear main air-guiding wall;
wherein the at least one lateral auxiliary air-guiding wall has a rear auxiliary air-introducing channel, and the rear auxiliary air-introducing channel has a rear auxiliary air-introducing opening perpendicular or inclined to the rear main air-guiding wall;
wherein the plurality of front main air-guiding walls are sequentially connected to form an outer peripheral surface having a circular or a polygonal surface profile, a nozzle opening of the second hollow tube body is shaped as a circle or an ellipse, and a radial distance of the rear main air-guiding wall is gradually decreased from the first hollow tube body to the second hollow tube body.

9. The nebulizer assembly according to claim 6,
wherein the air-guiding structure includes a plurality of front main air-guiding walls, a plurality of rear main air-guiding walls, and a plurality of first connection walls, the plurality of front main air-guiding walls are separate from each other and surroundingly disposed on the first hollow tube body, each of the plurality of rear main air-guiding walls is connected between a corresponding one of the plurality of front main air-guiding walls and the second hollow tube body, and each of the plurality of first connection walls is connected between the first hollow tube body and the second hollow tube body, and is connected between two adjacent ones of the rear main air-guiding walls;
wherein each of the plurality of rear main air-introducing channels is formed between a corresponding one of the plurality of front main air-guiding walls and the first hollow tube body, and each of the plurality of rear main air-introducing openings of each of the plurality of rear main air-introducing channels is perpendicular or inclined to the first hollow tube body.

10. The nebulizer assembly according to claim 6,
wherein the air-guiding structure includes a plurality of rear main air-guiding walls, and a plurality of first connection walls, each of the plurality of rear main air-guiding walls is connected between the first hollow tube body and the second hollow tube body, and each of the plurality of first connection walls is connected between the first hollow tube body and the second hollow tube body, and is connected between two adjacent ones of the plurality of rear main air-guiding walls;
wherein the air-guiding part has a rear air-guiding channel formed therein, each of the plurality of rear main air-introducing channels is formed between one of the plurality of rear main air-guiding walls and one of the plurality of rear air-guiding channel corresponding to each other, and each of the plurality of rear main air-introducing openings of each of the rear main air-introducing channels is perpendicular or inclined to the first hollow tube body;
wherein the first hollow tube body has a plurality of lateral main air-introducing openings separate from each other, each of the plurality of lateral main air-introducing openings extends along an outer peripheral surface of the first hollow tube body, and the plurality of lateral main air-introducing openings are respectively communicated with the plurality of rear main air-introducing openings;
wherein the air-guiding structure includes a plurality of second connection walls separate from each other, and each of the plurality of second connection walls is connected between two adjacent ones of the plurality of rear main air-guiding walls, and is adjacent to a corresponding one of the plurality of first connection walls;
wherein the air-guiding structure includes a surrounding extending wall surrounding the plurality of lateral main air-introducing openings, and the surrounding extending wall has a plurality of lateral auxiliary air-introducing openings respectively corresponding to the plurality of lateral main air-introducing openings.
